(19) United States
(12) Patent Application Publication
(10) Pub. No.: US 2024/0268690 A1
(43) Pub. Date: Aug. 15, 2024

(54) PORTABLE BLOOD PRESSURE SENSOR AND SENSING METHOD THEREOF

(71) Applicant: City University of Hong Kong, Hong Kong (HK)

(72) Inventors: Xinge YU, Hong Kong (HK); Jian LI, Hong Kong (HK); Huiling JIA, Hong Kong (HK); Jingkun ZHOU, Hong Kong (HK)

(21) Appl. No.: 18/163,891

(22) Filed: Feb. 3, 2023

(51) Int. Cl. A61B 5/0225; A61B 5/00; A61B 5/021

(52) U.S. Cl. CPC A61B 5/0225; A61B 5/02108; A61B 5/7278

(57) ABSTRACT

A portable blood pressure sensor comprises a sensing device, a force generation device, a first processor, a first flexible layer, and a second flexible layer. The sensing device comprises a first piezoelectric layer, and a second piezoelectric layer. The force generation device provides back pressure to the first and second piezoelectric layers. The first processor is electrically connected to the sensing device and the force generation device. The second flexible layer encapsulates the sensing device, the force generation device, and the first processor on the first flexible layer.

### FIELD OF THE INVENTION

The present invention generally relates to a portable sensor. More specifically the present invention relates to a portable blood pressure sensor.

### BACKGROUND OF THE INVENTION

Conventionally, blood pressure is measured with a manual cuff, in which a pressure gauge is equipped to measure the air pressure inside the cuff. By inflating the manual cuff to a pressure that is higher than BP, systolic BP and diastolic BP can be recorded with the assistance of the stethoscope. Typically, a trained medical professional is required during the measurement. Therefore, cuff-based oscillometry was developed to conduct the BP measurement automatically. In addition, applanation tonometry has been utilized to determine BP by measuring the pressure on skin generated by the contraction and expansion of an artery underneath the skin. However, only isolated, casual BP is measured in all these methods, which suffer from limited accuracy in reflecting BP over an extended time period.

### SUMMARY OF THE INVENTION

It is an objective of the present invention to provide a wireless, self-powered, integrated, miniaturized sensor capable of continuously monitoring of a BP pattern at home without the need for professional medical equipment, such as those provided in hospitals.

In accordance with a first aspect of the present invention, a portable blood pressure sensor comprises a sensing device, a force generation device, a first processor, a first flexible layer, and a second flexible layer. The sensing device comprises a first piezoelectric layer, and a second piezoelectric layer. The force generation device provides back pressure to the first and second piezoelectric layers. The first processor is electrically connected to the sensing device and the force generation device. The second flexible layer encapsulates the sensing device, the force generation device, and the first processor on the first flexible layer.

In accordance with one embodiment of the present invention, the first piezoelectric layer maintains a fixed distance from the second piezoelectric layer.

In accordance with another embodiment of the present invention, the fixed distance between the first and the second piezoelectric layers ranges from 14.5 mm to 15.5 mm.

In accordance with another embodiment of the present invention, the sensing device comprises a first bottom electrode, a second bottom electrode, a first top electrode, and a second top electrode. The first piezoelectric layer is located between the first bottom electrode and the first top electrode. The second piezoelectric layer is located between the second bottom electrode and the second top electrode.

In accordance with another embodiment of the present invention, the force generation device comprises a micro pump, a first airbag, and a second airbag. The first airbag is disposed above the first piezoelectric layer. The second airbag is disposed above the second piezoelectric layer. The first airbag is connected to the micro pump, and the second airbag is connected to the micro pump. The micro pump is configured to pump up the first airbag and the second airbag.

In accordance with another embodiment of the present invention, pressure in the first and second micro airbags ranges from 0 to 12 kPa.

In accordance with another embodiment of the present invention, diameters of the first and second micro airbags range from 5.5 to 6.5 mm, and thicknesses of the first and second micro airbags range from 0.5 to 1.5 mm.

In accordance with another embodiment of the present invention, the first piezoelectric layer generates a first piezo response data to the first processor, and the first processor generates a continuous pulse wave data according to the first piezo response data.

In accordance with another embodiment of the present invention, the first piezoelectric layer generates a first piezo response data to the first processor, and the second piezoelectric layer generates a second piezo response data to the first processor. The first processor generates a pulse wave velocity data according to the first and second piezo response data.

In accordance with a second aspect of the present invention, a blood pressure measuring system comprises the portable blood pressure sensor, a second processor, a display, and a third processor. The second processor is wirelessly connected to the first processor. The display is electrically connected to the second processor. The third processor is wirelessly connected to the second processor. The first processor generates a first data, and the second processor receives the first data. The second processor generates a second data according to the first data, and the third processor receives the second data. The third processor generates a blood pressure data from the second data through a XGBoost based data model. The second processor receives the blood pressure data, and the display shows a blood pressure information according to the blood pressure data.

In accordance with one embodiment of the present invention, the first data includes a continuous pulse wave data.

In accordance with one embodiment of the present invention, the first data includes a pulse wave velocity data.

In accordance with a third aspect of the present invention, a blood pressure measuring method comprises: providing back pressure to a first piezoelectric layer and a second piezoelectric layer; receiving a first piezo response data and a second piezo response data from the first piezoelectric layer and the second piezoelectric layer respectively; generating a continuous waveform and a localized pulse wave velocity (PWV) from the first piezo response data and the second piezo response data; and generating a predicted BP pattern from the continuous waveform and the localized PWV through a XGBoost based data model.

In accordance with one embodiment of the present invention, the step of providing the back pressure comprises: pumping up a first micro airbag and a second micro airbag. The first micro airbag is disposed above the first piezoelectric layer, and the second micro airbag is disposed above the second piezoelectric layer.

In accordance with another embodiment of the present invention, the step of generating the predicted BP pattern comprises: segmenting the continuous waveform to beat-to-beat waveform; extracting relevant features from the beat-to-beat waveform; and inputting the relevant features to the XGBoost based data model. The relevant features comprise systolic peak time, dicrotic notch time, diastolic peak time, and foot point.

In accordance with another embodiment of the present invention, the step of generating the predicted BP pattern comprises: inputting physiological factors to the XGBoost based data model. The physiological factors comprise heart rate, age, gender, and BMI.

### BRIEF DESCRIPTION OF THE DRAWINGS

Embodiments of the invention are described in more details hereinafter with reference to the drawings, in which:

FIG. 1 depicts an exploded view of a portable blood pressure sensor of some embodiments of the present invention;

FIG. 2 depicts a schematic view of a blood pressure measuring system of some embodiments of the present invention;

FIG. 3 depicts an exploded view of a sensing device of some embodiments of the present invention;

FIG. 4 depicts an exploded view of a data processing module of some embodiments of the present invention;

FIG. 5 depicts a flow chart of a blood pressure measuring method of some embodiments of the present invention;

FIG. 6 depicts a schematic view of a blood pressure measuring system of some embodiments of the present invention; and

FIG. 7 depicts a flow chart of preprocessing in the blood pressure measuring method of some embodiments of the present invention.

### DETAILED DESCRIPTION

In the following description, devices, systems, and measuring methods of blood pressure measuring and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

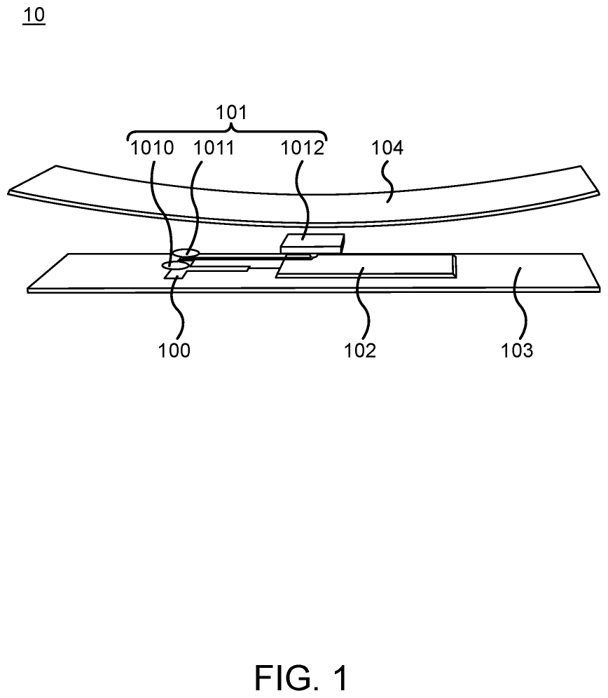

FIG. 1 depicts an exploded view of a portable blood pressure sensor of some embodiments of the present invention. In accordance with a first aspect of the present invention, the portable blood pressure (BP) sensor 10 comprises a sensing device 100, a force generation device 101, a data processing module 102, a first flexible layer 103, and a second flexible layer 104. The sensing device 100 is electrically connected to the data processing module 102, and the force generation device 101 is electrically connected to the data processing module 102. The force generation device 101 is disposed on the sensing device 100 and the data processing module 102. The second flexible layer 104 encapsulates the sensing device 100, the force generation device 101, and the data processing module 102 on the first flexible layer 103.

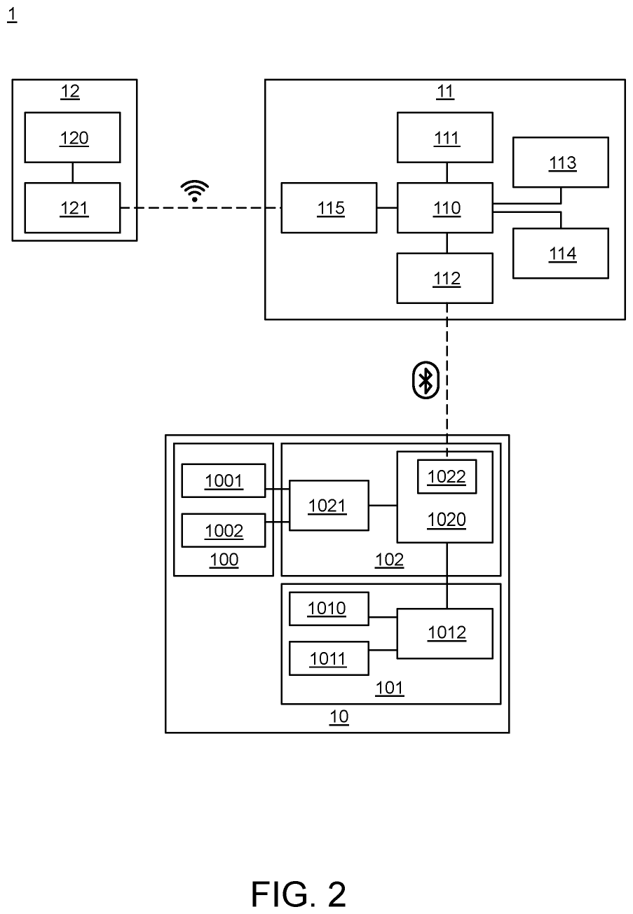

FIG. 2 depict a schematic view of a blood pressure measuring system of some embodiments of the present invention. Referring to FIG. 2, the sensing device 100 comprises first piezoelectric layer 1001 and second piezoelectric layer 1002, and the force generation device 101 provide back pressure to the first and second piezoelectric layers 1001, 1002.

The data processing module 102 comprises a first processor 1020, and the first processor 1020 is electrically connected to the sensing device 100 and the force generation device 101.

In this embodiment, the portable BP sensor 10 is flexible, and the portable BP sensor 10 can be worn as a wristband. While the portable BP sensor 10 is worn by a user, the first piezoelectric layer 1001 and the second piezoelectric layer 1002 can conformably contact with human artery. Therefore, the portable BP sensor 10 allows the conformable deformation of the first and second piezoelectric layers 1001, 1002 during the expansion and contraction of human artery. With the back pressure provided by the force generation device 101, the first processor 1020 can receive a piezo response from the first and second piezoelectric layers 1001, 1002 and generate first data that contains BP information.

For example, an exemplary dimension of the portable BP sensor 10 may be 15 cm×35 mm×4 mm, and, therefore, the portable BP sensor 10 can be worn on a user's wrist. The portable BP sensor 10 makes it possible for 24-hour ambulatory BP measurement at home, which can effectively reduce fatalities and medical costs of patients with cardiovascular and cerebrovascular diseases.

The BP measuring system 1 comprises the portable BP sensor 10, a second processor 110, a display 111, and a third processor 120. The second processor 110 is wirelessly connected to the first processor 1020, and the third processor 120 is wirelessly connected to the second processor 110.

In one aspect, the first processor 1020 can be a microcontroller (MCU), the second processor 110 can be a CPU of a portable device 11. For example, the portable device 11 can be, for example, but is not limited to, smart phone, or laptop. The third processor 120 can be a CPU of a server 12.

The first processor 1020 generates a first data, and the second processor 110 receives the first data. The second processor 110 generates a second data according to the first data, and the third processor 120 receives the second data. The server 12 is loaded with a XGBoost based data model. The third processor 120 generates a blood pressure data from the second data through the XGBoost based data model. The second processor 110 receives the BP data, and the display 111 shows a blood pressure information according to the blood pressure data. The BP information may include a predicted blood pressure pattern, systolic blood pressure, or diastolic blood pressure. Therefore, the BP measuring system 1 may automatically sense and record the BP information of a user through the portable BP sensor, so as to provide a 24-hour ambulatory BP measurement.

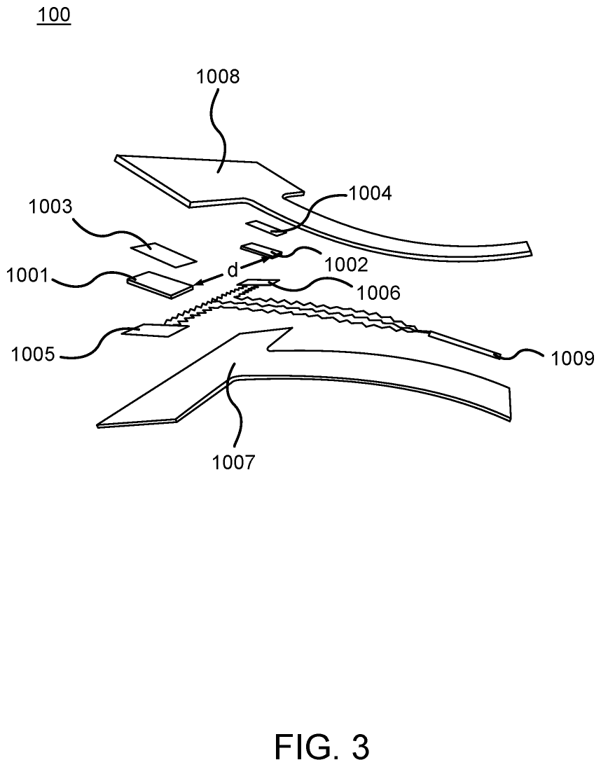

FIG. 3 depicts an exploded view of a sensing device of some embodiments of the present invention. Referring to FIG. 3, the first piezoelectric layer 1001 maintains a fixed distance d from the second piezoelectric layer 1002. Therefore, the first piezoelectric layer 1001 and the second piezoelectric layer 1002 can measure a localized pulse wave velocity (PWV) with the first and second piezoelectric layers 1001, 1002. In other words, the time difference of a single blood pulse waveform in arriving at two piezoelectric layers 1001, 1002 of the sensing device 100 is recorded to calculate the localized PWV in combination with the physical distance d between the two piezoelectric layers 1001, 1002. The localized PWV will be included in the first data generated by the first processer 1020 as shown in FIG. 2, and the localized PWV will be transmitted to the third processor 120 as shown in FIG. 2 for BP prediction, so as to improve the accuracy.

In one aspect, the distance d between the first and second piezoelectric layers 1001, 1002 is 15 mm. Therefore, the piezoelectric layers 1001, 1002 preserve sufficient physical distance, and the portable blood pressure sensor 10 can measure the localized PWV precisely with the piezoelectric layers 1001, 1002 of the sensing device 100. In some embodiments, the distance d between the first and second piezoelectric layers 1001, 1002 may range from 14.5 mm to 15.5 mm.

In this embodiment, the sensing device 100 has a first bottom electrode 1005, a second bottom electrode 1006, a first top electrode 1003, and a second top electrode 1004. The first piezoelectric layer 1001 is located between the first bottom electrode 1005 and the first top electrode 1003. The second piezoelectric layer 1002 is located between the second bottom electrode 1006 and the second top electrode 1004.

Moreover, the sensing device 100 comprises a connecting pin 1009. The connecting pin 1009 electrically connects the first and second bottom electrodes 1005, 1006 and the first processor 1020 as shown in FIG. 2. Consequently, one or more piezo responses are generated due to the potential difference across the sandwiched electrodes 1003, 1004, 1005, 1006 when suffering from mechanical deformation caused by blood propagation.

Furthermore, the sensing device 100 in this embodiment comprises a third flexible layer 1007 and a fourth flexible layer 1008. The fourth flexible layer 1008 encapsulates the electrodes 1003, 1004, 1005, 1006, and piezoelectric layers 1001, 1002 on the third flexible layer 1007, so as to provide a proper protection and maintain overall flexibility of the device.

Referring back to FIG. 1, the force generation device 101 comprises a micro pump 1012, a first micro airbag 1010, and a second micro airbag 1011. The first micro airbag 1010 is connected to the micro pump 1012, and the second micro airbag 1011 is connected to the micro pump 1012. The micro pump 1012 is disposed on the data processing module 102. The first processor 1020 as shown in FIG. 2 is electrically connected to the micro pump 1012, and, therefore; the first processor 1020 of the data processing module 102 can control the micro pump 1012.

The first micro airbag 1010 is disposed above the first piezoelectric layer 1001 (as shown in FIG. 3). The second micro airbag 1011 is disposed above the second piezoelectric layer 1002 (as shown in FIG. 3). The micro pump 1012 is configured to pump up the first micro airbag 1010 and the second micro airbag 1011. The first micro airbag 1010 can press the first piezoelectric layer 1001 against the first flexible layer 103, and the second micro airbag 1011 can press the second piezoelectric layer 1002 against the first flexible layer 103. Therefore, the micro pump 1012 can provide back pressure to the first piezoelectric layer 1001 and the second piezoelectric layer 1002 through the first micro airbag 1010 and the second micro airbag 1011, so as to measure the BP precisely.

In this embodiment, the pressure in the first micro airbag ranges from 0 to 12 kPa, and the pressure in the second micro airbag ranges from 0 to 12 kPa. Therefore, the first micro airbag 1010 and the second micro airbag 1011 can provide proper back pressure to the first piezoelectric layer 1001 and the second piezoelectric layer 1002 respectively.

Also, in this embodiment, diameters of the first and second micro airbags 1010, 1011 are 6 mm, and the thicknesses of the first and second micro airbags 1010, 1011 are 1 mm. Therefore, the first and second micro airbags 1010, 1011 can be installed in the BP measuring system 1. In some embodiments, the diameters of the first and second micro airbags 1010, 1011 range from 5.5 to 6.5 mm, and the thicknesses of the first and second micro airbags 1010, 1011 range from 0.5 to 1.5 mm.

Referring to FIG. 2, in the portable BP sensor 10, the data processing module 102 has an analog to digital convertor (ADC) 1021, and the ADC 1021 connects the sensing device 100 and the first processor 1020.

The piezo response from the first and second piezoelectric layers 1001, 1002 are converted to digital signal, and a first piezo response data and a second piezo response data are generated. The first processor 1020 processes the first and second piezo response data and generates the first data.

More particularly, the first piezoelectric layer 1001 generates a first piezo response data through the ADC 1021 to the first processor 1020, and the first processor 1020 generates a continuous pulse wave data according to the first piezo response data.

Also, the second piezoelectric layer 1002 generates a second piezo response data through the ADC 1021 to the first processor 1020, and the first processor 1020 generates a PWV data according to the first and second piezo response data.

In this embodiment, the first data includes the continuous pulse wave data and the PWV data. Therefore, the first data has sufficient information for BP prediction.

The first processor 1012 has a build-in Bluetooth module 1022, and the portable device 11 has a Bluetooth module 112. Therefore, the portable device 11 and the portable BP sensor 10 can be wirelessly connected through the Bluetooth module 112 and the Bluetooth module 1022, and the first data can be transmitted to the portable device 11.

In this embodiment, the portable device 11 has a denoise module 113 and a feature extraction module 114. After receiving the first data, the second processor 110 processes the first data. The processing include noise reduction and feature extraction, and relevant features will be generated from the continuous pulse wave data. The second processor 110 will generate the second data, and the second data includes the relevant features and the PWV data.

The portable device 11 has a Wi-Fi module 115, and the server 12 has a Wi-Fi module 121. The portable device 11 and the server 12 are wirelessly connected through the Wi-Fi modules 115, 121, and the second data can be transmitted to the server 12. In some embodiments, the portable device 11 and the server 12 can be wirelessly connected through mobile telecommunications such as 3G, 4G, 4.5G, or 5G.

XGBoost algorithm is proven effective for various classification and regression tasks in multiple fields. In this embodiment, the XGBoost based data model loaded in the server 12 is trained by experimental data of volunteers, and the result achieved an accuracy of −0.05±4.61 mmHg for systolic pressure, and 0.11±3.68 mmHg for diastolic pressure, respectively.

More particularly, this embodiment used the data collected from volunteers with each experiment duration more than 30 minutes. Before model training, the data was normalized in a range of 0 to 1. The training and validation set were randomly selected with a separation proportion of 4:1. The model evaluation is performed by five-fold cross-validation. The models were implemented using the Python package and library of scikit-learn.

The third processor 120 generates the BP data from the second data through the XGBoost based data model. The BP data is transmitted to the portable device 11 through the wireless connection between the Wi-Fi modules 115, 121, and the display 111 may show the BP information according to the BP data. Therefore, the BP measuring system can sense and record BP information of a user automatically in daily life.

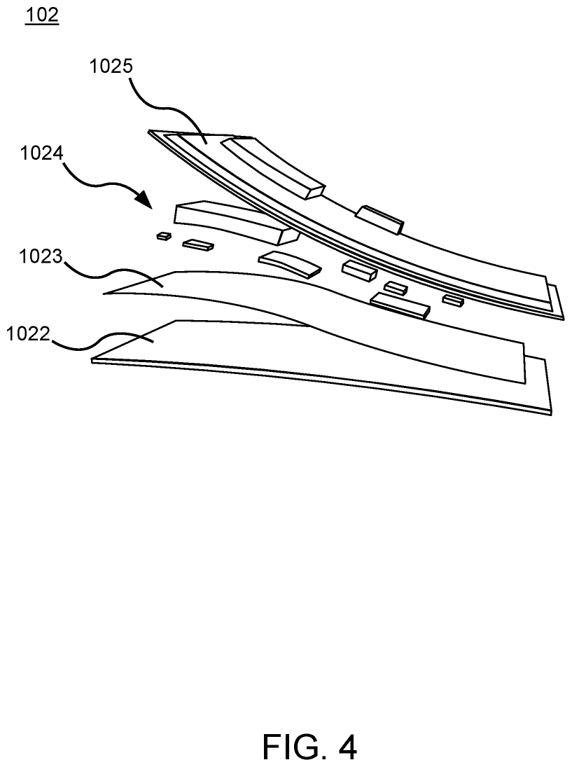

FIG. 4 depicts an exploded view of the data processing module 102 of some embodiments of the present invention. Referring to FIG. 4, the data processing module 102 comprises a fifth flexible layer 1022, a flexible printed circuit (FPC) 1023, electronic components 1024, and sixth flexible layer 1025.

The electronic components 1024 include the first processor 1020 and ADC 1021 as shown in FIG. 2, and the electronic components 1024 are disposed on the FPC 1023. The sixth flexible layer 1025 encapsulates the electronic components 1024 and the FPC 1023 on the fifth flexible layer 1022. Therefore, the data processing module 102 is flexible and well-protected, and the portable BP sensor 10 installed with the data processing module 102 can be worn on a user's wrist.

In the embodiments of the present invention, the first flexible layer 103, the second flexible layer 104, the third flexible layer 1007, the fourth flexible layer 1008, the fifth flexible layer 1022, and the sixth flexible layer 1025 may be made of silicone. Therefore, the portable BP sensor 10 can be easily and comfortably worn by a user.

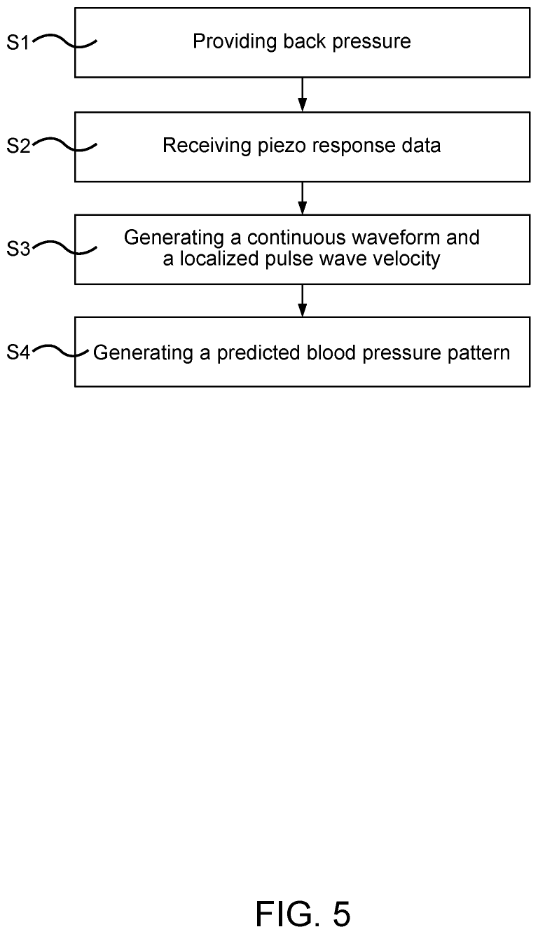

FIG. 5 depicts a flow chart of a blood pressure measuring method of some embodiments of the present invention. Referring to FIG. 5, in this embodiment, the BP measuring method provide back pressure to the first piezoelectric layer 1001 and the second piezoelectric layer 1002 (as shown in FIG. 3) (Step S1). This step provides the back pressure and close loop feedback for the sensing device 100 as shown in FIG. 1 to effectively detect the arterial deformation generated by blood propagation.

More particularly, the step of providing the back pressure comprises: pumping up the first micro airbag 1010 and the second micro airbag 1011 as shown in FIG. 1. The first micro airbag 1010 is disposed above the first piezoelectric layer 1001 as shown in FIG. 3, and the second micro airbag 1011 is disposed above the second piezoelectric layer 1002. Therefore, while the micro pump 1012 inflates the first micro airbag 1010 and the second micro airbag 1011, the first and second piezoelectric layers 1001, 1002 can be provided with back pressure respectively.

Referring to FIG. 5, after the back pressure is provided, the BP pressure measuring method of this embodiment receives the first piezo response data and the second piezo response data from the first piezoelectric layer 1001 and the second piezoelectric layer 1002 (as shown in FIG. 3) respectively (Step S2).

The step may comprise: receiving a first analogue piezo response data and a second analogue piezo response data from the first piezoelectric layer 1001 and the second piezoelectric layer 1002 respectively; and converting the first and second analogue piezo response data to the first and second piezo response data respectively. The conversion is executed by the ADC 1021 as shown in FIG. 2.

Referring to FIG. 5, after the first and second piezo response data are received, the measuring method generates a continuous waveform and the localized PWV from the first piezo response data and the second piezo response data (Step S3).

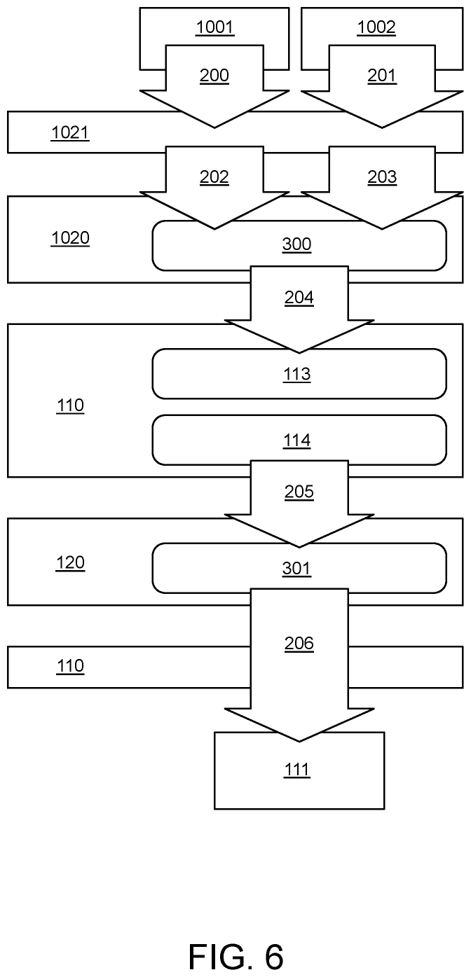

FIG. 6 depicts a schematic view of the BP measuring system of some embodiments of the present invention. Referring to FIGS. 5 and 6, the first analogue piezo response data 200 is converted to the first piezo response data 202 by the ADC 1021, and the second analogue piezo response data 201 is converted to the second piezo response data 203 by the ADC 1021, and the first and second piezo response data 202, 203 are digital data. The first and second piezo response data 202, 203 are transmitted to the first processor 1020.

The first processor 1020 is loaded with a mathematical model 300. The first processor 1020 generates the first data 204 according to the first and second piezo response data 202, 203 through the mathematical model 300.

The mathematical model 300 processes the piezo response data with the equation:

$$F ( t ) = {\int }_{0}^{t} V ( τ ) d τ ,$$

wherein the l′(t) corresponds to the output voltage of the piezoelectric layers 1001, 1002, and the F(t) corresponds to the force applied by the artery. Based on the mathematical model 300, the measured piezo response from radial artery and branchial artery was converted into BP pulse waveform. The first data 204 includes the continuous waveform and the localized PWV, and the continuous waveform includes the BP pulse waveform.

Referring to FIG. 5, after the continuous waveform and the localized PWV are generated, the BP measuring method generates a predicted BP pattern from the continuous waveform and the localized PWV through the XGBoost based data model (Step S4).

Referring to FIG. 6, before generating the predicted BP pattern, the BP measuring method executes a preprocessing of the continuous waveform. The first data 204 is transmitted to the second processor 110, and the denoise module 113 performs noise reduction processing on the continuous waveform of the first data 204. After noise reduction, the feature extraction module 114 performs feature extraction on the continuous waveform, and the relevant features are generated. The second data 205 including the relevant features and the localized PWV is transmitted to the third processor 120.

The third processor 120 is loaded with the XGBoost based data model 301, and the second data 205 is input into the XGBoost based data model 301, so as to output the blood pressure data 206. The blood pressure data 206 is transmitted back to the second processor 110 and the display shows the BP information, and the BP information includes the predicted BP pattern, the systolic BP and diastolic BP. In other words, the blood pressure data 206 contains information of the predicted BP pattern, the systolic BP and the diastolic BP.

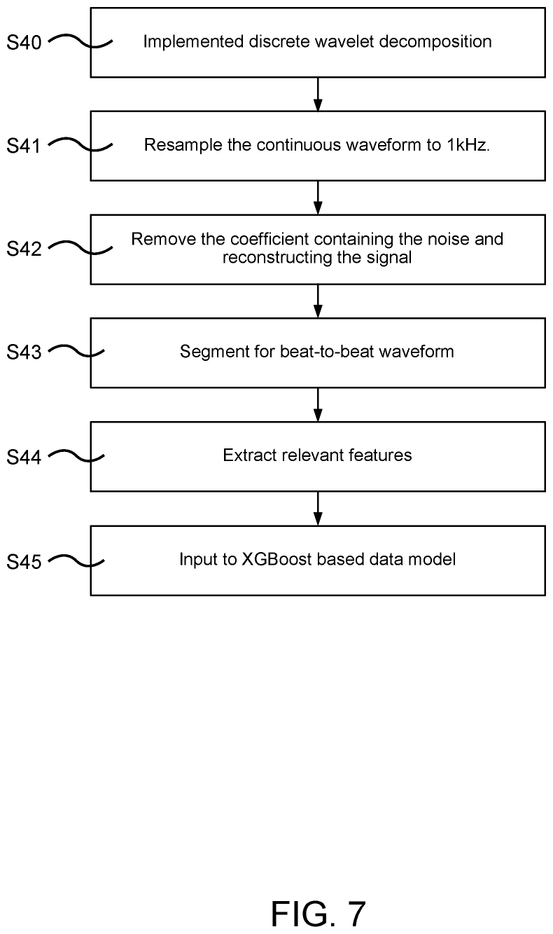

FIG. 7 depicts a flow chart of the preprocessing. Referring to FIG. 7, in this embodiment, the preprocessing of the continuous waveform comprises: performing implemented discrete wavelet decomposition (Step S40); resampling the continuous waveform to 1 kHz (Step S41); removing the coefficient containing the noise and reconstructing the signal (Step S42); segmenting for beat-to-beat waveform (Step S43); extracting relevant features (Step S44); and inputting to XGBoost based data model (Step S45). The steps S40, S41, S42, S43, S44 will be performed by the second processor 110 of the portable device 11. Also, to eliminate point detection errors, the method applied features cleaning to filter out mistaken data after Step S44, and the data differing too much from the empirical values will be removed before Step S45.

The step S4 of generating the predicted BP pattern comprises: segmenting the continuous wave to beat-to-beat waveform (Step S43); and extracting relevant features from the beat-to-beat waveform (Step S44); and inputting the relevant features to the XGBoost based data model (Step S45). The relevant features comprise systolic peak time, dicrotic notch time, diastolic peak time, and foot point. Therefore, the BP can be predicted precisely.

In some embodiments, the relevant features may comprise systolic peak time, dicrotic notch time, diastolic peak time, foot point, pulse peak-to-peak interval, pulse transit time between two pulse waves, 10% of systolic time span, 25% of systolic time span, 33% of systolic time span, 50% of systolic time span, 66% of systolic time span, 75% of systolic time span, 10% of diastolic time span, 25% of diastolic time span, 33% of diastolic time span, 50% of diastolic time span, 66% of diastolic time span, 75% of diastolic time span, Systolic peak height, Diastolic peak height, Relative augmentation index, Inflection point area ratio.

Furthermore, in some embodiments, the step of generating the predicted BP pattern (Step S4) may comprise: inputting physiological factors to the XGBoost based data model. The physiological factors comprise heart rate, age, gender, and BMI. Therefore, the BP can be predicted precisely with these factors.

The functional units and modules of the sensors, systems, and/or methods in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A portable blood pressure sensor, comprising:
a sensing device comprising:
a first piezoelectric layer; and
a second piezoelectric layer;
a force generation device providing back pressure to the first and second piezoelectric layers;
a first processor electrically connected to the sensing device and the force generation device;
a first flexible layer; and
a second flexible layer;
wherein the second flexible layer encapsulates the sensing device, the force generation device, and the first processor on the first flexible layer.

2. The portable blood pressure sensor of claim 1, wherein the first piezoelectric layer maintains a fixed distance from the second piezoelectric layer.

3. The portable blood pressure sensor of claim 2, wherein the fixed distance between the first and second piezoelectric layers ranges from 14.5 mm to 15.5 mm.

4. The portable blood pressure sensor of claim 1, wherein the sensing device comprises:
a first bottom electrode;
a second bottom electrode;
a first top electrode; and
a second top electrode;
wherein the first piezoelectric layer is located between the first bottom electrode and the first top electrode, and the second piezoelectric layer is located between the second bottom electrode and the second top electrode.

5. The portable blood pressure sensor of claim 1, wherein the force generation device comprises:
a micro pump;
a first micro airbag disposed above the first piezoelectric layer; and
a second micro airbag disposed above the second piezoelectric layer;
wherein the first micro airbag is connected to the micro pump, and the second micro airbag is connected to the micro pump,
wherein the micro pump is configured to pump up the first micro airbag and the second micro airbag.

6. The portable blood pressure sensor of claim 5, wherein pressure in the first and second micro airbags ranges from 0 to 12 kPa.

7. The portable blood pressure sensor of claim 5, wherein diameters of the first and second micro airbags range from 5.5 to 6.5 mm, and thicknesses of the first and second micro airbags range from 0.5 to 1.5 mm.

8. The portable blood pressure sensor of claim 1, wherein the first piezoelectric layer generates a first piezo response data to the first processor, and the first processor generates a continuous pulse wave data according to the first piezo response data.

9. The portable blood pressure sensor of claim 1, wherein the first piezoelectric layer generates a first piezo response data to the first processor, and the second piezoelectric layer generates a second piezo response data to the first processor, and the first processor generates a pulse wave velocity data according to the first and second piezo response data.

10. A blood pressure measuring system, comprising:
a portable blood pressure sensor as set forth in claim 1;
a second processor wirelessly connected to the first processor;
a display electrically connected to the second processor; and
a third processor wirelessly connected to the second processor;
wherein the first processor generates a first data, and the second processor receives the first data;
wherein the second processor generates a second data according to the first data, and the third processor receives the second data;
wherein the third processor generates a blood pressure data from the second data through a XGBoost based data model, and the second processor receives the blood pressure data, and the display shows a blood pressure information according to the blood pressure data.

11. The blood pressure measuring system of claim 10, wherein the first data includes a continuous pulse wave data.

12. The blood pressure measuring system of claim 10, wherein the first data includes a pulse wave velocity data.

13. A blood pressure measuring method, comprising:
providing back pressure to a first piezoelectric layer and a second piezoelectric layer;
receiving a first piezo response data and a second piezo response data from the first piezoelectric layer and the second piezoelectric layer respectively;
generating a continuous waveform and a localized pulse wave velocity (PWV) from the first piezo response data and the second piezo response data; and
generating a predicted BP pattern from the continuous waveform and the localized PWV through a XGBoost based data model.

14. The blood pressure measuring method of claim 13, wherein the step of providing the back pressure comprises:
pumping up a first micro airbag and a second micro airbag;
wherein the first micro airbag is disposed above the first piezoelectric layer, and the second micro airbag is disposed above the second piezoelectric layer.

15. The blood pressure measuring method of claim 13, wherein the step of generating the predicted BP pattern comprises:
segmenting the continuous waveform to beat-to-beat waveform;
extracting relevant features from the beat-to-beat waveform; and
inputting the relevant features to the XGBoost based data model;
wherein the relevant features comprise systolic peak time, dicrotic notch time, diastolic peak time, and foot point.

16. The blood pressure measuring method of claim 13, wherein the step of generating the predicted BP pattern comprises:
Inputting physiological factors to the XGBoost based data model;
wherein the physiological factors comprise heart rate, age, gender, and BMI.
